(19) United States
(12) Patent Application Publication
(10) Pub. No.: US 2025/0025447 A1
(43) Pub. Date: Jan. 23, 2025

(54) TOLL-LIKE RECEPTOR AGONISTS AND ANTAGONISTS AND USES THEREOF

(71) Applicant: Regents of the University of Minnesota, Minneapolis, MN (US)

(72) Inventor: David M. FERGUSON, Minneapolis, MN (US)

(21) Appl. No.: 18/705,175

(22) PCT Filed: Nov. 2, 2022
(86) PCT No.: PCT/US2022/048745

§ 371 Date: Apr. 26, 2024

Related U.S. Application Data
(60) Provisional application No. 63/275,378, filed on Nov. 3, 2021.

(51) Int. Cl. A61K 31/437; A61K 45/06; A61P 37/06; C07D 471/04

(52) U.S. Cl. CPC A61K 31/437; A61K 45/06; A61P 37/06; C07D 471/04

(57) ABSTRACT

The present disclosure provides compounds of Formula I which can be tuned to provide agonistic or antagonistic toll-like receptor (TLR) bining properties. Further provided herein are pharmaceutical compositions comprising compounds described herein, as well as methods of using such compositions to treat a disease or condition in a subject.

### CROSS REFERENCE TO RELATED APPLICATION

This application claims priority from U.S. Provisional Application No. 63/275,378, filed Nov. 3, 2021, which is hereby incorporated by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BACKGROUND OF THE DISCLOSURE

Toll-like receptor (TLR) modulators can be used in various therapeutic settings, e.g., as vaccine adjuvants. However, many factors have limited the efficacy of TLR modulating compounds such as imiquimod. As an example, although such compounds can induce proinflamatory cytokines, they may also concurrently induce significant levels of anti-inflammatory cytokines such as IL-10. Thus, there exists an unmet need to develop novel TLR modulating compounds that can trigger a more desirable ratio of pro- to anti-inflammatory cytokines and provide both agonistic and antagonist receptor binding.

### SUMMARY OF THE DISCLOSURE

The present disclosure provides compounds with agonistic and/or antagonistic toll-like-receptor binding properties. In some embodiments, provided herein are imidazoquinoline compounds with various substitutents on the C2-position, as well as methods of evaluating and using such compounds. In some cases, the C2-substitutions can be isomeric substitutions, e.g., those of a hexyl, pentyl, or butyl group.

In various embodiments, the imidazoquinoline compounds of the present disclosure can be a compound of Formula I:

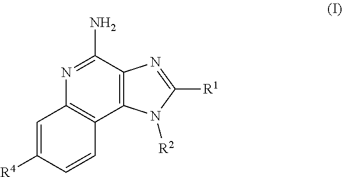

wherein,
R¹, R², and R⁴ are each independently (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)-cycloalkyl, aryl, heteroaryl, or heterocycle, which can be optionally substituted with one or more groups independently selected from the group consisting of halo, cyano, hydroxy, oxo, oxiranyl, (C₃-C₅)cycloalkyl, optionally substituted aryl, and optionally substituted heteroaryl,
or a pharmaceutically acceptable salt thereof, and
wherein, when R⁴ is —C(═O)OCH₃ and R² is —CH₂(p-Phe)—CH₂NH₂, R¹ is not —CH₂—CH₂—CH₂—CH₃.

In some embodiments, R¹ is optionally substituted (C₁-C₆)alkyl. In certain embodiments, R¹ is CH₂—CH(Me)₂ or

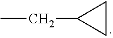

In some aspects, R² is optionally substituted —CH₂—CH(OH)Me or —CH₂(p-Phe)—CH₂NH₂.

In further embodiments, R⁴ is —C(═O)O—(C₁-C₄)alkyl.

In certain aspects, the compound is selected from the group consisting of:

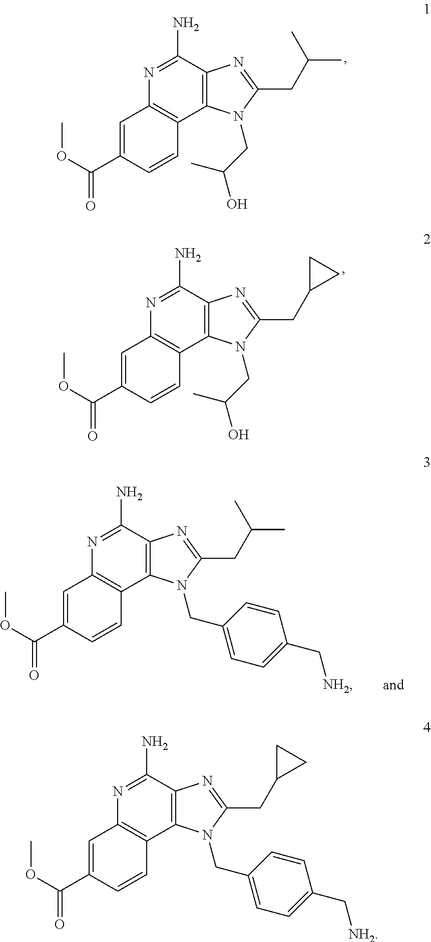 [SMILES: COC(=O)c1ccc2c(c1)nc(N)c1nc(CC(C)C)n(CC(C)O)c12 ; COC(=O)c1ccc2c(c1)nc(N)c1nc(CC3CC3)n(CC(C)O)c12 ; COC(=O)c1ccc2c(c1)nc(N)c1nc(CC(C)C)n(Cc3ccc(CN)cc3)c12 ; "and" ; COC(=O)c1ccc2c(c1)nc(N)c1nc(CC3CC3)n(Cc3ccc(CN)cc3)c12]

In various aspects, the present disclosure provides pharmaceutical compositions comprising a compound of Formula I and a pharmaceutically acceptable diluent or carrier.

In certain aspects, the present disclosure provides methods of modulating a TLR-7 or TLR-8 receptor, the methods comprising contacting the TLR-7 or TLR-8 receptor with a compound of the present dislcoure. In some embodiments, the methods comprise administering to the subject a compound of Formula I or a pharmaceutically acceptable salt thereof, or a pharmaceutical composition thereof

In some embodiments, the present disclosure provides methods of stimulating an immune response in a subject, the methods comprising administering to the subject a compound of Formula I, or a pharmaceutically acceptable salt thereof, or a pharmaceutical composition thereof.

In various embodiments, the present disclosure provides methods of treating a cancer in a subject in need thereof, the methods comprising administering to the subject a compound of Formula I or a pharmaceutically acceptable salt thereof, or a pharmaceutical composition thereof. In some embodiments, the cancer is multiple myeloma, pancreatic cancer, or lung cancer.

In some aspects, the present disclosure provides methods of treating an autoimmune disease or condition in a subject in need thereof, the methods comprising administering to the subject a compound of Formula I, or a pharmaceutically acceptable salt thereof, or a pharmaceutical composition thereof.

In various embodiments, the present disclosure provides methods of treating an inflammatory disease or condition in a subject in need thereof, the method comprising administering to the subject a compound of Formula I, or a pharmaceutically acceptable salt thereof, or a pharmaceutical composition thereof.

### DESCRIPTION OF THE FIGURES

The novel features of the invention are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIGS. 1A-D illustrates a graph showing HEK-SEAP reporter cell measurements for determining agonistic and/or antagonist TLR binding properties. This assay measured the NF-κB downstream signal mediated by the activation of myeloid differentiation factor 88 (MyD88) by agonist binding.

FIGS. 2A-D show dose-response curves for imidazolequinoline derivatives, compounds 1-4, at TLR-7.

FIGS. 3A-D show dose-response curves for imidazolequinoline derivatives, compounds 1-4, at TLR-8.

FIGS. 4A-C show antagonist dose response curves for compound 3 and illustrates the use of compound 3 as a potent inhibitor of TNFα (FIG. 4A), IFNγ (FIG. 4B), and IL-1β production (FIG. 4C).

### DETAILED DESCRIPTION

### I. Introduction

The present disclosure provides compounds having agonistic and/or antagonistic toll-like-receptor (TLR) binding properties. The compounds of the present disclosure can be used for the treatment of cancer and infectious diseases. The activation of these receptors results in an immunostimulatory effect through the production of proinflammatory cytokines, such as tumor necrosis factor (TNF) and various interleukins (TL), and the anti-viral type I interferons (IFNs).

In some cases, a compound of this disclosure is a TLR agonist. In other aspects, a compound herein is a TLR antagonist. A TLR to which the compounds of this disclosure may bind include TLR-7, TLR-8, and related TLRs. Further provided herein are surprising structural determinators that may provide a compound with an agonistic or antagonist TLR binding profile.

In an example, a C-7 substituent other than H and such as an ester may drive a compound's antagonistic function for a TLR. Furthermore, and as further described herein, a substituent at the C-2 position of the thiazoquinoline scaffold can provide a compound with antagonistic TLR binding properties.

Number ranges are to be understood as inclusive, i.e., including the indicated lower and upper limits. Furthermore, the term “about”, as used herein, and unless clearly indicated otherwise, generally refers to and encompasses plus or minus 10% of the indicated numerical value(s). For example, “about 10%” may indicate a range of 9% to 11%, and “about 1” may include the range 0.9-1.1.

It is noted that as used herein and in the appended claims, the singular forms “a”, “an”, and “the” include plural reference unless the context clearly dictates otherwise. Thus, for example, reference to “a compound” includes a plurality of such compounds and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim.

“Subject” as used herein refers to an individual to which an ADC or TLR-7/8 agonist, as described herein, is administered. Examples of a “subject” include, but are not limited to, a mammal such as a human, rat, mouse, guinea pig, non-human primate, pig, goat, cow, horse, dog, cat, bird, and fowl. Typically, a subject is a rat, mouse, dog, non-human primate, or human. In some aspects, the subject is a human.

The term “optionally substituted,” refers to an indicated group being either substituted or unsubstituted.

The term “antibody” as used herein covers intact monoclonal antibodies, polyclonal antibodies, monospecific antibodies, multispecific antibodies (e.g., bispecific antibodies), including intact antibodies and antigen binding antibody fragments, and reduced forms thereof in which one or more of the interchain disulfide bonds are disrupted, that exhibit the desired biological activity and provided that the antigen binding antibody fragments have the requisite number of attachment sites for the desired number of attached groups, such as a linker (L), as described herein. In some aspects, the linkers are attached via a succinimide or hydrolyzed succinimide to the sulfur atoms of cysteine residues of reduced interchain disulfide bonds and/or cysteine residues introduced by genetic engineering. The native form of an antibody is a tetramer and consists of two identical pairs of immunoglobulin chains, each pair having one light chain and one heavy chain. In each pair, the light and heavy chain variable domains (VL and VH) are together primarily responsible for binding to an antigen. The light chain and heavy chain variable domains consist of a framework region interrupted by three hypervariable regions, also called “complementarity determining regions” or “CDRs.” The light chain and heavy chains also contain constant regions that may be recognized by and interact with the immune system. The antibody is derivable from any suitable species. In some aspects, the antibody is of human or murine origin, and in some aspects the antibody is a human, humanized or chimeric antibody. Antibodies can be fucosylated to varying extents or afucosylated.

An “antigen” is an entity to which an antibody specifically binds.

All terms, chemical names, expressions, and designations have their usual meanings which are well-known to those skilled in the art. When a group of substituents is disclosed herein, it is understood that all individual members of that group and all subgroups, including any isomers, enantiomers, and diastereomers of the group members, are disclosed separately.

When a Markush group or other grouping is used herein, all individual members of the group and all combinations and subcombinations possible of the group are intended to be individually included in the disclosure. When a compound is described herein such that a particular isomer, enantiomer or diastereomer of the compound is not specified, for example, in a formula or in a chemical name, that description is intended to include each isomers and enantiomer of the compound described individually or in any combination. Additionally, unless otherwise specified, all isotopic variants of compounds disclosed herein are intended to be encompassed by the disclosure. Specific names of compounds are intended to be exemplary, as it is known that one of ordinary skill in the art can name the same compounds differently.

As used herein, the term “group” may refer to a reactive functional group of a chemical compound. Groups of the present compounds refer to an atom or a collection of atoms that are a part of the compound. Groups of the present disclosure may be attached to other atoms of the compound via one or more covalent bonds. Groups may also be characterized with respect to their valence state. The present disclosure includes groups characterized as monovalent, divalent, trivalent, etc. valence states.

As used herein, the term “substituted” refers to a compound (e.g., an alkyl chain) wherein a hydrogen is replaced by another reactive functional group or atom, as described herein.

As used herein, a broken line in a chemical structure can be used to indicate a bond to the rest of the molecule. For example,

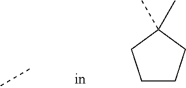

is used to designate the 1-position as the point of attachment of 1-methylcyclopentate to the rest of the molecule. Alternatively,

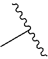

in, e.g.,

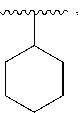 [SMILES: *C1CCCCC1]

can be used to indicate that the given moiety, the cyclohexyl moiety in this example, is attached to a molecule via the bond that is “capped” with the wavy line.

The following definitions are used, unless otherwise described: halo is fluoro, chloro, bromo, or iodo. Alkyl, alkoxy, alkenyl, alkynyl, etc. denote both straight and branched groups; but reference to an individual radical such as propyl embraces only the straight chain radical, a branched chain isomer such as isopropyl being specifically referred to. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(X) wherein X is absent or is H, O, (C₁-C₄)alkyl, phenyl or benzyl, as well as a radical of an ortho-fused bicyclic heterocycle of about eight to ten ring atoms comprising one to four heteroatoms each selected from the group consisting of non-peroxide oxygen, sulfur, and N(X).

It will be appreciated by those skilled in the art that compounds of the disclosure having a chiral center may exist in and be isolated in optically active and racemic forms. Some compounds may exhibit polymorphism. It is to be understood that the present disclosure encompasses any racemic, optically-active, polymorphic, or stereoisomeric form, or mixtures thereof, of a compound of the disclosure, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₃-C₆)cycloalkyl(C₁-C₆)alkyl can be cyclopropylmethyl, cyclobutylmethyl, cyclopentylmethyl, cyclohexylmethyl, 2-cyclopropylethyl, 2-cyclobutylethyl, 2-cyclopentylethyl, or 2-cyclohexylethyl; (C₁-C₆)alkoxy can be methoxy, ethoxy, propoxy, isopropoxy, butoxy, iso-butoxy, sec-butoxy, pentoxy, 3-pentoxy, or hexyloxy; (C₂-C₆)alkenyl can be vinyl, allyl, 1-propenyl, 2-propenyl, 1-butenyl, 2-butenyl, 3-butenyl, 1,-pentenyl, 2-pentenyl, 3-pentenyl, 4-pentenyl, 1-hexenyl, 2-hexenyl, 3-hexenyl, 4-hexenyl, or 5-hexenyl; (C₂-C₆)alkynyl can be ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, or 5-hexynyl; (C₁-C₆)alkanoyl can be acetyl, propanoyl or butanoyl; (C₁-C₆)alkoxycarbonyl can be methoxycarbonyl, ethoxycarbonyl, propoxycarbonyl, isopropoxycarbonyl, butoxycarbonyl, pentoxycarbonyl, or hexyloxycarbonyl; (C₂-C₆)alkanoyloxy can be acetoxy, propanoyloxy, butanoyloxy, isobutanoyloxy, pentanoyloxy, or hexanoyloxy; aryl can be phenyl, indenyl, or naphthyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term “alkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) and is derived by the removal of one hydrogen atom from the parent alkane. Representative straight chain “C₁-C₈ alkyl” groups include, but are not limited to, methyl, ethyl, n-propyl, n-butyl, n-pentyl, n-hexyl, n-heptyl and n-octyl; while branched C₁-C₈ alkyls include, but are not limited to, isopropyl, sec-butyl, isobutyl, tert-butyl, isopentyl, and 2-methylbutyl.

The term “alkylene” refers to a bivalent unsubstituted saturated branched or straight chain hydrocarbon of the stated number of carbon atoms (e.g., a C₁-C₆ alkylene has from 1 to 6 carbon atoms) and having two monovalent centers derived by the removal of two hydrogen atoms from the same or two different carbon atoms of the parent alkane. Alkylene groups can be substituted with 1-6 fluoro groups, for example, on the carbon backbone (as —CHF— or —CF₂—) or on terminal carbons of straight chain or branched alkylenes (such as —CHF₂ or —CF₃). Alkylene groups include but are not limited to: methylene (—CH₂—), ethylene (—CH₂CH₂—), n-propylene (—CH₂CH₂CH₂—), n-propylene (—CH₂CH₂CH₂—), n-butylene (—CH₂CH₂CH₂CH₂—), difluoro-methylene (—CF₂—), tetrafluoroethylene (—CF₂CF₂—), and the like.

The term “alkenyl” refers to an unsubstituted straight chain or branched, hydrocarbon having at least one carbon-carbon double bond and the indicated number of carbon atoms (e.g., “C₂-C₈ alkenyl” or “C₂-C₁₀” alkenyl have from 2 to 8 or 2 to 10 carbon atoms, respectively). When the number of carbon atoms is not indicated, the alkenyl group has from 2 to 6 carbon atoms.

The term “heteroalkyl” refers to a stable straight or branched chain saturated hydrocarbon having the stated number of total atoms and at least one (e.g., 1 to 15) heteroatom selected from the group consisting of O, N, Si and S. The carbon and heteroatoms of the heteroalkyl group can be oxidized (e.g., to form ketones, N-oxides, sulfones, and the like) and the nitrogen atoms can be quaternized. The heteroatom(s) can be placed at any interior position of the heteroalkyl group and/or at any terminus of the heteroalkyl group, including termini of branched heteroalkyl groups), and/or at the position at which the heteroalkyl group is attached to the remainder of the molecule. Heteroalkyl groups can be substituted with 1-6 fluoro groups, for example, on the carbon backbone (as —CHF— or —CF₂—) or on terminal carbons of straight chain or branched heteroalkyls (such as —CHF₂ or —CF₃). Examples of heteroalkyl groups include, but are not limited to, —CH₂—CH₂—O—CH₃, —CH₂—CH₂—NH—CH₃, —CH₂—CH₂—N(CH₃)₂, —C(═O)—NH—CH₂—CH₂—NH—CH₃, —C(═O)—N(CH₃)—CH₂—CH₂—N(CH₃)₂, —C(═O)—NH—CH₂—CH₂—NH—C(═O)—CH₂—CH₃, —C(═O)—N(CH₃)—CH₂—CH₂—N(CH₃)—C(═O)—CH₂—CH₃, —O—CH₂—CH₂—CH₂—NH(CH₃), —O—CH₂—CH₂—CH₂—N(CH₃)₂, —O—CH₂—CH₂—CH₂—NH—C(═O)—CH₂—CH₃, —O—CH₂—CH₂—CH₂—N(CH₃)—C(═O)—CH₂—CH₃, —CH₂—CH₂—CH₂—NH(CH₃), —O—CH₂—CH₂—CH₂—N(CH₃)₂, —CH₂—CH₂—CH₂—NH—C(═O)—CH₂—CH₃, —CH₂—CH₂—CH₂—N(CH₃)—C(═O)—CH₂—CH₃, —CH₂—S—CH₂—CH₃, —CH₂—CH₂—S(O)—CH₃, —NH—CH₂—CH₂—NH—C(═O)—CH₂—CH₃, —CH₂—CH₂—S(O)₂—CH₃, —CH₂—CH₂—O—CF₃, and —Si(CH₃)₃. Up to two heteroatoms may be consecutive, such as, for example, —CH₂—NH—OCH₃ and —CH₂—O—Si(CH₃)₃. A terminal polyethylene glycol (PEG) moiety is a type of heteroalkyl group.

The term “alkynyl” refers to an unsubstituted straight chain or branched, hydrocarbon having at least one carbon-carbon triple bond and the indicated number of carbon atoms (e.g., “C₂-C₈ alkynyl” or “C₂-C₁₀” alkynyl have from 2 to 8 or 2 to 10 carbon atoms, respectively). When the number of carbon atoms is not indicated, the alkynyl group has from 2 to 6 carbon atoms.

The term “acyl” refers to an alkyl, haloalkyl, alkenyl, alkynyl, aryl cycloalkyl, heteroaryl, or heterocyclyl group, as defined herein, connected to the remainder of the compound by a C═O (carbonyl) group.

The term “carboxamido” refers to a —C(═O)NRR′ group, wherein R and R′ are independently selected from the group consisting of hydrogen, alkyl, alkenyl, alkynyl, aryl cycloalkyl, heteroaryl, and heterocyclyl, as defined herein.

The term “heteroalkylene” refers to a bivalent unsubstituted straight or branched group derived from heteroalkyl (e.g., as defined herein). Examples of heteroalkylene groups include, but are not limited to, —CH₂—CH₂—O—CH₂—, —CH₂—CH₂—O—CF₂—, —CH₂—CH₂—NH—CH₂—, —C(═O)—NH—CH₂—CH₂—NH—CH₂—, —C(═O)—N(CH₃)—CH₂—CH₂—N(CH₃)—CH₂—, —C(═O)—NH—CH₂—CH₂—NH—C(═O)—CH₂—CH₂—, —C(═O)—N(CH₃)—CH₂—CH₂—N(CH₃)—C(═O)—CH₂—CH₂—, —O—CH₂—CH₂—CH₂—NH—CH₂—, —O—CH₂—CH₂—CH₂—N(CH₃)—CH₂—, —O—CH₂—CH₂—CH₂—NH—C(═O)—CH₂—CH₂—, —O—CH₂—CH₂—CH₂—N(CH₃)—C(═O)—CH₂—CH₂—, —CH₂—CH₂—CH₂—NH—CH₂—, —CH₂—CH₂—CH₂—N(CH₃)—CH₂—, —CH₂—CH₂—CH₂—NH—C(═O)—CH₂—CH₂—, —CH₂—CH₂—CH₂—N(CH₃)—C(═O)—CH₂—CH₂—, —CH₂—CH₂—NH—C(═O)—, —CH₂—CH₂—N(CH₃)—CH₂—, —CH₂—CH₂—N(CH₃)₂—, —NH—CH₂—CH₂(NH₂)—CH₂—, and —NH—CH₂—CH₂(NHCH₃)—CH₂—. A bivalent polyethylene glycol (PEG) moiety is a type of heteroalkylene group.

The term “alkoxy” refers to an alkyl group, as defined herein, which is attached to a molecule via an oxygen atom. For example, alkoxy groups include, but are not limited to methoxy, ethoxy, n-propoxy, iso-propoxy, n-butoxy, sec-butoxy, tert-butoxy, n-pentoxy and n-hexoxy.

The term “alkylthio” refers to an alkyl group, as defined herein, which is attached to a molecule via a sulfur atom. For example, alkythio groups include, but are not limited to thiomethyl, thioethyl, thio-n-propyl, thio-iso-propyl, and the like.

The term “haloalkyl” refers to an unsubstituted straight chain or branched, saturated hydrocarbon having the indicated number of carbon atoms (e.g., “C₁-C₄ alkyl,” “C₁-C₆ alkyl,” “C₁-C₈ alkyl,” or “C₁-C₁₀” alkyl have from 1 to 4, to 6, 1 to 8, or 1 to 10 carbon atoms, respectively) wherein at least one hydrogen atom of the alkyl group is replaced by a halogen (e.g., fluoro, chloro, bromo, or iodo). When the number of carbon atoms is not indicated, the haloalkyl group has from 1 to 6 carbon atoms. Representative C₁₋₆ haloalkyl groups include, but are not limited to, trifluoromethyl, 2,2,2-trifluoroethyl, and 1-chloroisopropyl.

The term “cycloalkyl” refers to a cyclic, saturated, or partially unsaturated hydrocarbon having the indicated number of carbon atoms (e.g., “C₃₋₈ cycloalkyl” or “C₃₋₆” cycloalkyl have from 3 to 8 or 3 to 6 carbon atoms, respectively). When the number of carbon atoms is not indicated, the cycloalkyl group has from 3 to 6 carbon atoms. Cycloalkyl groups include bridged, fused, and spiro ring systems, and bridged bicyclic systems where one ring is aromatic and the other is unsaturated. Representative “C₃₋₆ cycloalkyl” groups include, cyclopropyl, cyclobutyl, cyclopentyl, and cyclohexyl.

The term “aryl” refers to an unsubstituted monovalent carbocyclic aromatic hydrocarbon group of 6-10 carbon atoms derived by the removal of one hydrogen atom from a single carbon atom of a parent aromatic ring system. Aryl groups include, but are not limited to, phenyl, naphthyl, anthracenyl, biphenyl, and the like.

The term “heterocycle” refers to a saturated or partially unsaturated ring or a multiple condensed ring system, including bridged, fused, and spiro ring systems. Heterocycles can be described by the total number of atoms in the ring system, for example a 3-10 membered heterocycle has 3 to 10 total ring atoms. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl and piperidinyl. The term “heterocycle” also includes multiple condensed ring systems (e.g., ring systems comprising 2, 3 or 4 rings) wherein a single heterocycle ring (as defined above) can be condensed with one or more heterocycles (e.g., decahydronapthyridinyl), carbocycles (e.g., decahydroquinolyl) or aryls. The rings of a multiple condensed ring system can be connected to each other via fused, spiro and bridged bonds when allowed by valency requirements. It is to be understood that the point of attachment of a multiple condensed ring system (as defined above for a heterocycle) can be at any position of the multiple condensed ring system including a heterocycle, aryl and carbocycle portion of the ring. It is also to be understood that the point of attachment for a heterocycle or heterocycle multiple condensed ring system can be at any suitable atom of the heterocycle or heterocycle multiple condensed ring system including a carbon atom and a heteroatom (e.g., a nitrogen). Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl, tetrahydrothiopyranyl, 1,2,3,4-tetrahydroquinolyl, benzoxazinyl, dihydrooxazolyl, chromanyl, 1,2-dihydropyridinyl, 2,3-dihydrobenzofuranyl, 1,3-benzodioxolyl, and 1,4-benzodioxanyl.

The term “heteroaryl” refers to an aromatic hydrocarbon ring system with at least one heteroatom within a single ring or within a fused ring system, selected from the group consisting of O, N and S. The ring or ring system has 4n+2 electrons in a conjugated π system where all atoms contributing to the conjugated π system are in the same plane. In some embodiments, heteroaryl groups have 5-10 total ring atoms and 1, 2, or 3 heteroatoms (referred to as a “5-10 membered heteroaryl”). Heteroaryl groups include, but are not limited to, imidazole, triazole, thiophene, furan, pyrrole, benzimidazole, pyrazole, pyrazine, pyridine, pyrimidine, and indole.

The term “hydroxyl” refers to an —OH group. The term “cyano” refers to a —CN group.

The term “carboxy” refers to a —C(═O)OH group. The term “oxo” refers to a═O group.

The term “alkanoyl” refers to an alkyl group, as defined herein, connected to the remainder of the molecule by a —C(═O) group. Exemplary alkanoyl groups include, but are not limited to acetyl, n-propanoyl, and n-butanoyl.

The term “alkanoyloxy” refers to an alkyl group, as defined herein, connected to the remainder of the molecule by an —OC(═O) group. Exemplary alkanoyloxy groups include, but are not limited to acetoxy, n-propanoyloxy, and n-butanoyloxy.

The term “alkoxycarbonyl” refers to an alkoxy group, as defined herein, connected to a C(═O)-alkyl group via the oxygen atom of the alkoxy (i.e., an alkyl ester group).

The terms “arylalkyl” and “cycloalkylalkyl” refer to an aryl group or a cycloalkyl group (as defined herein) connected to the remainder of the molecule by an alkyl group, as defined herein. Exemplary arylalkyl groups include but are not limited to benzyl and phenethyl.

Exemplary cycloalkylalkyl groups include, but are not limited to cyclopropylmethyl, cyclobutylmethyl, cyclopentylethyl, and cyclohexylethyl.

The term “amino acid,” as used herein, comprises the residues of the natural amino acids (e.g., Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g., phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, a-methyl-alanine, para-benzoylphenylalanine, phenylglycine, propargylglycine, sarcosine, and tert-butylglycine). The term also comprises natural and unnatural amino acids bearing a conventional amino protecting group (e.g., acetyl or benzyloxycarbonyl), as well as natural and unnatural amino acids protected at the carboxy terminus (e.g., as a (C₁-C₆)alkyl, phenyl or benzyl ester or amide; or as an a-methylbenzyl amide). Other suitable amino and carboxy protecting groups are known to those skilled in the art (See for example, T. W. Greene, Protecting Groups In Organic Synthesis; Wiley: New York, 1981, and references cited therein). An amino acid can be linked to the remainder of a compound of formula I through the carboxy terminus, the amino terminus, or through any other convenient point of attachment, such as, for example, through the sulfur of cysteine.

The term “peptide,” as used herein, describes a sequence of at least about 2 and not more than about 25 amino acids and/or peptidyl residues. The peptide sequence may be linear or cyclic. As an example, a cyclic peptide can be prepared or may result from the formation of disulfide bridges between two cysteine residues in a sequence. A peptide can be linked to the remainder of a compound of Formula I through the carboxy terminus, the amino terminus, or through any other convenient point of attachment, such as, for example, through the sulfur of a cysteine. In some cases, a peptide herein comprises 3 to 25, 5 to 21, or 10 to 25 amino acids. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

As used herein, the term “free drug” refers to a biologically active drug molecule (e.g., one of Formula I) that is not covalently attached to another moiety, such as a peptide or protein. Accordingly, free drug refers to a compound as it exists immediately upon cleavage from a conjugate, e.g., a drug-peptide or drug-protein conjugate as described herein. The release mechanism can be via a cleavable linker in the conjugate, or via intracellular conversion or metabolism of the conjugate. In some aspects, the free drug will be protonated and/or may exist as a charged moiety. The free drug is a pharmacologically active species which is capable of exerting a particular biological effect. In some embodiments, the pharmacologically active species is the parent drug alone. In some embodiments, the pharmacologically active species is the parent drug bonded to another molecule, e.g., in a conjugate.

The terms “treat” or “treatment,” unless otherwise indicated or implied by context, refer to therapeutic treatment and prophylactic measures to prevent relapse, wherein the object is to inhibit an undesired physiological change or disorder, such as, for example, the development or spread of cancer. For purposes of the present disclosure, beneficial or desired clinical results include, but are not limited to, alleviation of symptoms, diminishment of extent of disease, stabilized (e.g., not worsening) state of disease, delay or slowing of disease progression, amelioration or palliation of the disease state, and remission (whether partial or total), whether detectable or undetectable. “Treatment,” in some aspects, also includes prolonging survival as compared to expected survival if not receiving treatment.

In the context of cancer, the term “treating” includes any or all of: inhibiting growth of cancer cells or of a tumor; inhibiting replication of cancer cells, lessening of overall tumor burden or decreasing the number of cancer cells, and ameliorating one or more symptoms associated with the disease.

As to any of the above groups that contain one or more substituents, it is understood that such groups do not contain any substitution or substitution patterns which are sterically impractical and/or synthetically non-feasible. In addition, as further described herein, the compounds of this disclosure can include all stereochemical isomers (and racemic mixtures) arising from the substitution of these compounds.

### II. TLR-Modulating Compounds

Provided herein are compounds having agonistic and/or antagonistic toll-like-receptor binding properties. In some cases, a compound of this disclosure is a TLR agonist. In other aspects, a compound herein is a TLR antagonist. A TLR to which the compounds of this disclosure may bind include TLR-7, TLR-8, etc.

### Compound Structure

In some embodiments, provided herein is a compound of Formula I:

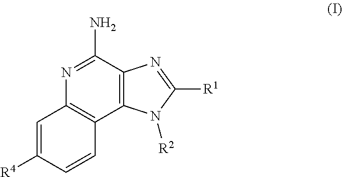

wherein:
R¹, R², and R⁴ are independently (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)-cycloalkyl, aryl, heteroaryl, or heterocycle, which can be optionally substituted with one or more groups independently selected from the group consisting of halo, cyano, hydroxy, oxo, oxiranyl, (C₃-C₅)cycloalkyl, optionally substituted aryl, and optionally substituted heteroaryl,
or a pharmaceutically acceptable salt thereof, and
wherein, when R⁴ is —C(═O)OCH₃ and R² is —CH₂(p-Phe)—CH₂NH₂, R¹ is not —CH₂—CH₂—CH₂—CH₃.

In some embodiments, R¹ is optionally substituted (C₁-C₆)alkyl. In further embodiments, R¹ is CH₂—CH(Me)₂ or

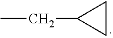

In some aspects, R² is optionally substituted —CH₂—CH(OH)Me or —CH₂(p-Phe)—CH₂NH₂.

In some embodiments, R¹ is optionally substituted (C₁-C₆)alkyl. In such cases, R¹ can be —CH₂—CH(Me)₂ or CH₂—CH(Me)₂ or

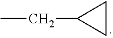

R² is optionally substituted —CH₂—CH(OH)Me and —CH₂(p-Phe)—CH₂NH₂.

In some embodiments, a compound of the present disclosure, e.g., a compound of any one of Formula I, can be capable of binding to a toll-like receptor (TLR). In some embodiments, the binding of a compound to a TLR can exhibit an agonist effect on the TLR. In some embodiments, the binding of a compound to a TLR can exert an immunostimulatory effect.

In certain aspects, the compound is selected from the group consisting of:

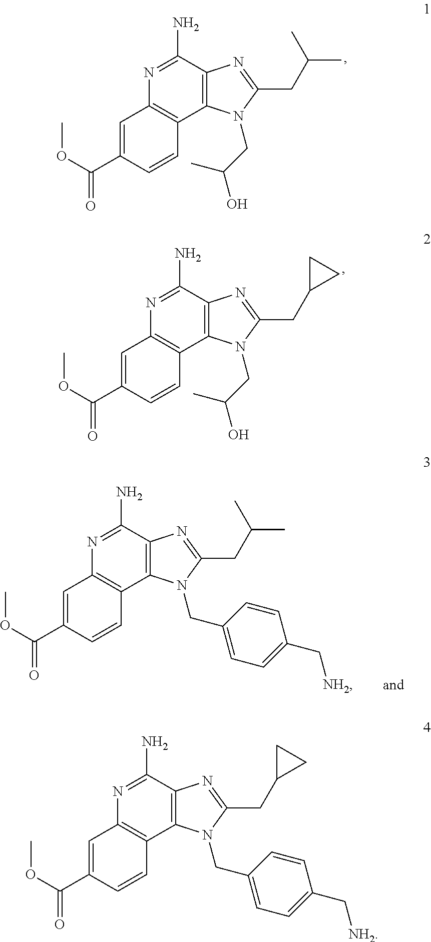 [SMILES: COC(=O)c1ccc2c(c1)nc(N)c1nc(CC(C)C)n(CC(C)O)c12 ; COC(=O)c1ccc2c(c1)nc(N)c1nc(CC3CC3)n(CC(C)O)c12 ; COC(=O)c1ccc2c(c1)nc(N)c1nc(CC(C)C)n(Cc3ccc(CN)cc3)c12 ; "and" ; COC(=O)c1ccc2c(c1)nc(N)c1nc(CC3CC3)n(Cc3ccc(CN)cc3)c12]

In certain aspects, the compound is selected from the group consisting of;

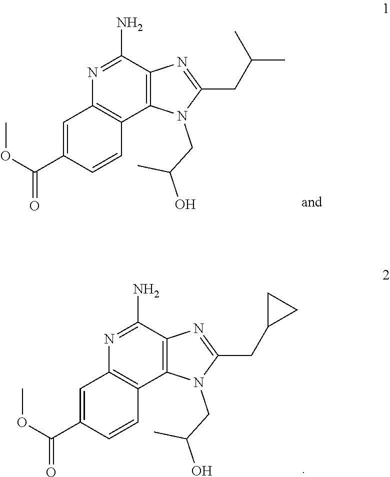 [SMILES: COC(=O)c1ccc2c(c1)nc(N)c1nc(CC(C)C)n(CC(C)O)c12 ; COC(=O)c1ccc2c(c1)nc(N)c1nc(CC3CC3)n(CC(C)O)c12]

In certain aspects, the compound is selected from the group consisting of:

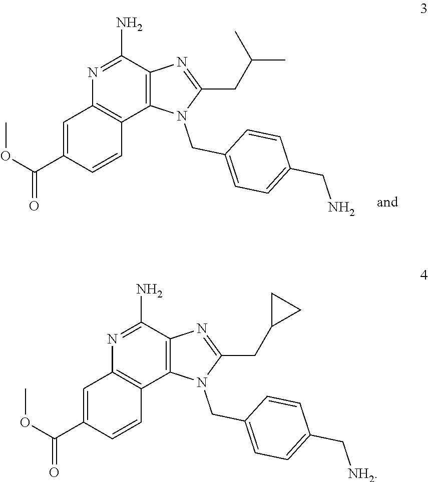 [SMILES: COC(=O)c1ccc2c(c1)nc(N)c1nc(CC(C)C)n(Cc3ccc(CN)cc3)c12 ; COC(=O)c1ccc2c(c1)nc(N)c1nc(CC3CC3)n(Cc3ccc(CN)cc3)c12]

In certain aspects, the compound is:

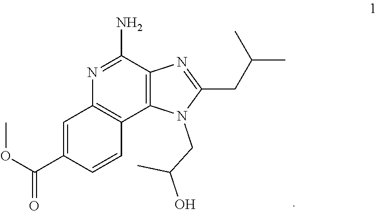 [SMILES: COC(=O)c1ccc2c(c1)nc(N)c1nc(CC(C)C)n(CC(C)O)c12]

In certain aspects, the compound is:

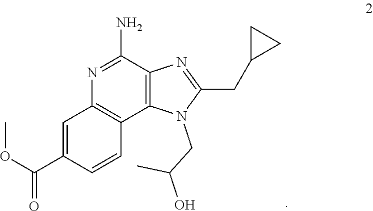 [SMILES: COC(=O)c1ccc2c(c1)nc(N)c1nc(CC3CC3)n(CC(C)O)c12]

In certain aspects, the compound is:

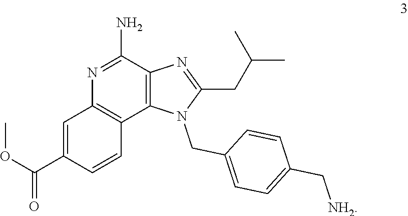 [SMILES: COC(=O)c1ccc2c(c1)nc(N)c1nc(CC(C)C)n(Cc3ccc(CN)cc3)c12]

In certain aspects, the compound is:

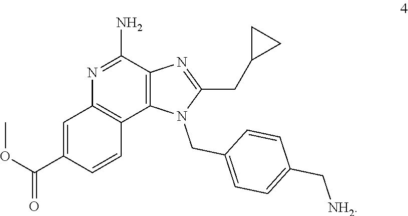 [SMILES: COC(=O)c1ccc2c(c1)nc(N)c1nc(CC3CC3)n(Cc3ccc(CN)cc3)c12]

### Compound Synthesis

Processes for preparing compounds of the present disclosure are also provided. In some embodiments, a compound described herein can be synthesized as shown below in SCHEME 1:

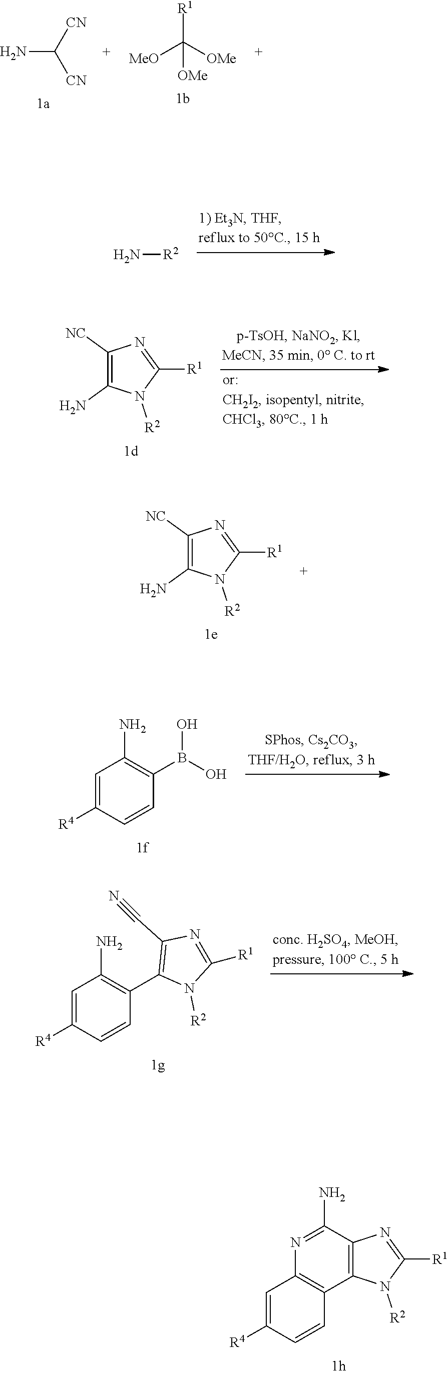

In some embodiments, R¹, R², and R⁴ are each independently (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)-cycloalkyl, aryl, heteroaryl, or heterocycle, which can be optionally substituted with one or more groups independently selected from the group consisting of halo, cyano, hydroxy, oxo, oxiranyl, (C₃-C₅)cycloalkyl, optionally substituted aryl, and optionally substituted heteroaryl, and when R⁴ is —C(═O)OCH₃ and R² is —CH₂(p-Phe)—CH₂NH₂, R¹ is not —CH₂—CH₂—CH₂—CH₃.

In some embodiments, R¹ is optionally substituted (C₁-C₆)alkyl. In such cases, R¹ can be —CH₂—CH(Me)₂ or

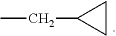

R²is optionally substituted —CH₂—CH(OH)Me or —CH₂(p-Phe)—CH₂NH₂.

### III. Conjugates

The present disclosure further provides conjugates comprising a TLR-binding compound of the present disclosure (e.g., a compound of Formula I) coupled to another molecule, such as a small molecule, a peptide, a protein, or a nucleic acid. In some cases, the TLR-binding compound is coupled to an antibody, a T cell receptor, or an antigen binding fragment (e.g., a single-chain variable fragment). In some cases, the antibody, the T cell receptor, or the antigen binding fragment thereof targets a cancer cell. In some cases, the TLR-binding compound is coupled to the molecule by a linker. In some cases, the linker is a cleavable linker.

### IV. Pharmaceutical Compositions

Provided herein are pharmaceutical compositions comprising a one or more compounds of Formula I and/or one or more conjugates according to the present disclosure. In some instances, administration of a compound of Formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, a-ketoglutarate, and a-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium, or lithium) or alkaline earth metal (for example, calcium) salts of carboxylic acids can also be made.

The compound(s) of Formula I can be formulated as pharmaceutical compositions and administered to a mammalian host, such as a human patient, in a variety of forms adapted to the chosen route of administration, e.g., orally or parenterally, by intravenous, intramuscular, topical or subcutaneous routes. Thus, the presently disclosed compounds may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations can contain at least 0.1% of active compound. The percentage of the compositions and preparations may be varied and may conveniently be between about 2% to about 60% of the weight of a given unit dosage form. The amount of active compound in such therapeutically useful compositions is such that an effective dosage level (e.g., when measured systemically and/or locally post-administration) will be obtained.

A tablet, troche, pill, capsule, and the like comprising one or more compounds of Formula I may further contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propyl parabens as preservatives, a dye and flavoring such as cherry or orange flavor. Generally, any material used in preparing any unit dosage form should be pharmaceutically acceptable and substantially non-toxic in the amounts employed. In addition, the active compound may be incorporated into sustained-release preparations and devices.

A compound of the present disclosure may also be administered intravenously or intraperitoneally by infusion or injection. Solutions of such compound(s) or its pharmaceutically acceptable salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid, and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or using surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions. For topical administration, the present compounds may be applied in pure form, e.g., when they are liquids. However, it will generally be desirable to administer them to the skin as compositions or formulations, in combination with a dermatologically acceptable carrier, which may be a solid or a liquid. Useful solid carriers include finely divided solids such as talc, clay, microcrystalline cellulose, silica, alumina, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user. Examples of useful dermatological compositions which can be used to deliver the compound(s) of Formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508). Useful dosages of the compound(s) of Formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, for use in treatment may vary not only with the particular salt selected but also with the route of administration, the nature of the condition being treated and the age and condition of the patient and will be ultimately at the discretion of the attendant physician or clinician. The desired dose may conveniently be presented in a single dose or as divided doses administered at appropriate intervals, for example, as two, three, four or more sub-doses per day. The sub-dose itself may be further divided, e.g., into several discrete loosely spaced administrations; such as multiple inhalations from an insufflator or by application of a plurality of drops into the eye.

The compositions comprising a compound of Formula I, a conjugate, and/or a pharmaceutically acceptable salt thereof, can be formulated in a unit dosage form, each dosage containing from about 5 to about 1,000 mg (1 g), more usually about 100 mg to about 500 mg, of the active ingredient. The term “unit dosage form” refers to physically discrete units suitable as unitary dosages for human subjects and other subjects, each unit containing a predetermined quantity of active material (i.e., a compound of Formula I, or a pharmaceutically acceptable salt thereof) calculated to produce the desired therapeutic effect, in association with a suitable pharmaceutical excipient.

An effective amount of the active material (i.e., a compound or conjugate of Formula I, or a pharmaceutically acceptable salt of any of the foregoing) is ordinarily supplied at a dosage level of from about 0.01 mg/kg to about 1000 mg/kg of body weight per day, or any range therein. In some cases, the range is from about 0.05 to about 500 mg/kg of body weight per day, or any range therein. In some cases, from about 0.1 to about 250 mg/kg of body weight per day, or any range therein. In some cases, from about 0.1 to about 100 mg/kg of body weight per day, or any range therein. In an example, the range can be from about 0.1 to about 50.0 mg/kg of body weight per day, or any amount or range therein. In another example, the range can be from about 0.01 to about 15.0 mg/kg of body weight per day, or any range therein. In yet another example, the range can be from about 0.05 to about 7.5 mg/kg of body weight per day, or any amount to range therein. In yet another example, the range can be from about 0.1 to about 5.0 mg/kg of body weight per day, or any amount to range therein. Pharmaceutical compositions comprising a compound of Formula I, or a pharmaceutically acceptable salt of any of the foregoing, can be administered on a regimen of 1 to 4 times per day or in a single daily dose.

### V. Methods of Use

Provided herein are methods of using one or more compounds of Formula I, pharmaceutically acceptable salts thereof, conjugate(s) thereof, and/or pharmaceutical compositions comprising such compounds for the treatment of a disease or condition in a subject in need thereof. In some embodiments, such methods can comprise administering to a subject in need thereof a compound of Formula I, a pharmaceutically acceptable salt thereof, a conjugate thereof, or pharmaceutical composition of this disclosure in a therapeutically effective amount to thereby treat such disease or condition in the subject. In some cases, the subject is a mammal such as a human or a rodent. Diseases and/or conditions that can be treated using the compounds of the present disclosure include but are not limited to cancer, autoimmune diseases or conditions, inflammatory diseases or conditions, and viral or bacterial infections.

TLR-7 and TLR-8 serve important roles in the innate immune system and are important therapeutic targets. Both TLR-7 and TLR-8 have been implicated in many conditions, including autoimmune diseases and conditions. Their activation by self-antigens can lead to an inflammatory response and production of self-reactive T-cells and antibodies, thereby contributing to the pathogenesis of numerous autoimmune diseases. For example, systemic lupus erythematosus (SLE) is one autoimmune disease linked to TLR-7, where TLR-7 is believed to play a role in lupus pathogenesis specifically through the induction of IFN-α by plasmacytoid dendritic cells. TLR-7 expression also correlates with increased disease activity in rheumatoid arthritis, another autoimmune disease. In this case, it is believed ssRNA in synovial fluid acts as a TLR-7 ligand.

While TLR activation in immune cells may produce an antitumor effect, the activation of TLRs within tumor cells themselves may promote tumor growth. The overexpression of TLR-7 and TLR-8 has been seen in various malignancies including multiple myeloma, pancreatic cancer, and lung cancer. Stimulation of these receptors by agonists in human lung cancer tumor cells lead to increased tumor cell survival and resistance to therapeutics. Thus, TLR-7 and TLR-8 play an important role in autoimmune diseases, inflammation, and tumor cell regulation.

Provided herein are methods of modulating an immune response in a subject in need thereof, the method comprising modulating a TLR-7 and/or TLR-8 receptor using the compounds of Formula I, pharmaceutically acceptable salts thereof, conjugate(s) thereof, and/or pharmaceutical compositions comprising such compounds.

In some aspects, compounds of Formula I activate the TLR-7 and/or TLR-8 receptors resulting in an immunostimulatory effect, including the production of proinflammatory cytokines, such as tumor necrosis factor (TNF) and various interleukins (TL), and the anti-viral type I interferons (IFNs). In some embodiments, the compounds of Formula I stimulate TNFα, IFNγ, and IL-1β production. These cytokines are of particular interest due to the role they play in the driving Th1 differentiation of immune cells and development of cellular immunity. In some embodiments, the compound of Formula I is a TLR7/8 agonist. In some cases, the compound of Formula I is compound 1, compound 2, or a combination thereof.

In some aspects, compounds of Formula I diminish TLR-7 and/or TLR-8 receptor activity, which can result in an immunosuppressive effect, and can diminish antibody and proinflammatory cytokine production as well as B cell activity. While TLR7/8 activation can promote innate and inflammatory immune responses essential for disease (e.g., viral infection and cancer) clearance, TLR7/8 overactivation can be a central cause of autoimmune activity, overinflammation, and abberant baseline effector function activity in many chronic diseases. As disclosed herein, (e.g., at EXAMPLE 7) certain compounds of Formula I can antagonize TLR7 and TLR8, and can therefore be helpful for managing a number of chronic conditions. In some embodiments, a method for treating a subject in need thereof comprises administering an imidazoquinoline compound which is a TLR7/8 antagonist. In some embodiments, a method for treating a subject in need thereof comprises administering a compound of Formula I to the subject which diminishes TLR7/8 activity. In some cases, the compound of Formula I is administered following an immunostimulatory therapy to return immune activity towards baseline (e.g., pre-treatment) levels for a subject. In some cases, the compound of Formula I is a TLR7/8 antagonist. In some cases, the compound of Formula I is compound 3, compound 4, or a combination thereof.

The methods provided herein comprise methods of treating one or more disease(s) in a subject in need thereof. Some embodiments provide a method of treating an autoimmune disease or condition or inflammatory disease or condition in a subject in need thereof, comprising administering to the subject a therapeutically effective amount a compound of Formula I, a pharmaceutically acceptable salt thereof, a conjugate thereof, or a pharmaceutical composition thereof. Some embodiments provide a method of inducing an immune regulatory response in a subject in need thereof, the method comprising administering to the subject a therapeutically effective amount a compound of Formula I, a pharmaceutically acceptable salt thereof, a conjugate thereof, or a pharmaceutical composition thereof. In some embodiments, the compound of Formula I is compound 3, compound 4, or a combination thereof. In some embodiments, the autoimmune or inflammatory disease or condition is systemic lupus erythematosus (SLE) or rheumatoid arthritis.

Some embodiments provide a method of treating an autoimmune disease or condition in a subject in need thereof, comprising administering to the subject a therapeutically effective amount a compound of Formula I, a pharmaceutically acceptable salt thereof, a conjugate thereof, or a pharmaceutical composition thereof in combination with another immune modulatory therapy. Compounds of Formula I can be administered to the subject before, during, or after administration of the immune modulatory therapy. In some embodiments, the compound of Formula I, a pharmaceutically acceptable salt thereof, a conjugate thereof, or a pharmaceutical composition thereof can be administered to the subject following treatment with immune modulatory therapy. In some cases, the compound of Formula I is a TLR7/8 agonist. In some cases, the compound of Formula I is a TLR7/8 antagonist. In some cases, the compound of Formula I is compound 1, compound 2, or a combination thereof. In some cases, the compound of Formula I is compound 3, compound 4, or a combination thereof. In some cases, the immune modulatory therapy comprises administering a glucocorticoid, a cytostatic agent, an antibody, an immunophilin modulator, a calcineurin modulator, an interferon, an interleukin, a cytokine, or a combination thereof.

Cancers, including, but not limited to, a tumor, metastasis, or other disease or disorder characterized by abnormal cells that are characterized by uncontrolled cell growth in some embodiments are treated or inhibited by administration of a compound of the present disclosure, or a conjugate thereof as disclosed herein. In some embodiments, the subject has previously undergone treatment for the cancer. In some embodiments, the prior treatment is surgery, radiation therapy, administration of one or more anticancer agents, or a combination of any of the foregoing.

The methods provided herein comprise methods of treating one or more disease or condition in a subject in need thereof. Some embodiments provide a method of treating cancer in a subject in need thereof, comprising administering to the subject a therapeutically effective amount a compound of Formula I, a pharmaceutically acceptable salt thereof, a conjugate thereof, or a pharmaceutical composition thereof. In various embodiments, the present disclosure provides methods of treating a cancer in a subject in need thereof, the methods comprising administering to the subject a compound of Formula I or a pharmaceutically acceptable salt thereof, or a pharmaceutical composition thereof. In some cases, the compound of Formula I is a TLR7/8 agonist. In some cases, the compound of Formula I is a TLR7/8 antagonist. In some cases, the compound of Formula I is compound 1, compound 2, or a combination thereof. In some cases, the compound of Formula I is compound 3, compound 4, or a combination thereof. In some embodiments, the cancer is multiple myeloma, pancreatic cancer, or lung cancer.

Some embodiments provide a method of inducing an anti-tumor immune response in a subject in need thereof, comprising administering to the subject a therapeutically effective amount a compound of Formula I, a pharmaceutically acceptable salt thereof, a conjugate thereof, or a pharmaceutical composition thereof. In some embodiments, the methods provided herein comprise methods of treating multiple myeloma, pancreatic cancer, or lung cancer in a subject in need thereof, comprising administering to the subject a therapeutically effective amount of a compound of Formula I, a pharmaceutically acceptable salt thereof, a conjugate thereof, or a pharmaceutical composition thereof. In some cases, the compound of Formula I is a TLR7/8 agonist. In some cases, the compound of Formula I is a TLR7/8 antagonist. In some cases, the compound of Formula I is compound 1, compound 2, or a combination thereof. In some cases, the compound of Formula I is compound 3, compound 4, or a combination thereof.

Some embodiments provide a method of treating cancer in a subject in need thereof, comprising administering to the subject a therapeutically effective amount a compound of Formula I, a pharmaceutically acceptable salt thereof, or a conjugate thereof in combination with another anticancer therapy (e.g., surgery and radiation therapy) and/or anticancer agent (e.g., an immunotherapy such as nivolumab or pembrolizumab). Compounds of Formula I can be administered to the subject before, during, or after administration of the anticancer therapy and/or anticancer agent. In some embodiments, the compounds of Formula I described herein can be administered to the subject following treatment with radiation and/or after surgery.

Some embodiments provide a method for delaying or preventing acquired resistance to an anticancer agent, comprising administering to the subject a therapeutically effective amount a compound of Formula I, a pharmaceutically acceptable salt thereof, or a conjugate thereof to a patient at risk for developing or having acquired resistance to an anticancer agent. In some cases, the compound of Formula I is a TLR7/8 agonist. In some cases, the compound of Formula I is a TLR7/8 antagonist. In some cases, the compound of Formula I is compound 1, compound 2, or a combination thereof. In some cases, the compound of Formula I is compound 3, compound 4, or a combination thereof. In some embodiments, the patient is administered a dose of the anticancer agent (e.g., at substantially the same time as a dose of the compound of Formula I, or a salt thereof is administered to the patient).

Some embodiments provide a method of delaying and/or preventing development of cancer resistant to an anticancer agent in a subject, comprising administering to the subject a therapeutically effective amount a compound of Formula I, a pharmaceutically acceptable salt thereof, or a conjugate thereof before, during, or after administration of a therapeutically effective amount of the anticancer agent. In some cases, the compound of Formula I is a TLR7/8 agonist. In some cases, the compound of Formula I is a TLR7/8 antagonist. In some cases, the compound of Formula I is compound 1, compound 2, or a combination thereof. In some cases, the compound of Formula I is compound 3, compound 4, or a combination thereof.

Compounds of Formula I, pharmaceutically acceptable salts thereof, conjugates thereof, and/or pharmaceutical compositions comprising such compounds, are useful for inhibiting the multiplication of a cancer cell, causing apoptosis in a cancer cell, for increasing phagocytosis of a cancer cell, and/or for treating cancer in a subject in need thereof. In some embodiments, the cancer is as described herein. In some embodiments, the subject has previously undergone treatment for the cancer. In some embodiments, the prior treatment is surgery, radiation therapy, administration of one or more anticancer agents, or a combination of any of the foregoing. In some embodiments, the subject has discontinued a prior therapy, for example, due to unacceptable or unbearable side effects, wherein the prior therapy was too toxic, or wherein the subject developed resistance to the prior therapy. Some embodiments provide a method for delaying or preventing a disease or disorder, comprising administering to the subject a therapeutically effective amount of a compound of Formula I, a pharmaceutically acceptable salt thereof, a conjugate thereof, and/or a pharmaceutical compositions comprising such compound, and a vaccine against the disease or disorder, to a patient at risk for developing the disease or disorder. In some embodiments, the disease or disorder is cancer, as described herein. In some embodiments, the disease or disorder is a viral pathogen. In some embodiments, the vaccine is administered subcutaneously. In some embodiments, the vaccine is administered intramuscularly. In some embodiments, the compound of Formula I, pharmaceutically acceptable salt thereof, conjugate thereof, and/or pharmaceutical compositions comprising such compound, and the vaccine are administered via the same route (for example, the compound of Formula I, pharmaceutically acceptable salt thereof, conjugate thereof, and/or pharmaceutical compositions comprising such compound, and the vaccine are both administered subcutaneously). In some embodiments, the compound of Formula I, pharmaceutically acceptable salt thereof, conjugate thereof, and/or pharmaceutical compositions comprising such compound, and the vaccine are administered via different routes. In some embodiments, the vaccine and the compound of Formula I, pharmaceutically acceptable salt thereof, conjugate thereof, and/or pharmaceutical compositions comprising such compound are provided in separate formulations. In some embodiments, the compounds of Formula I described herein are present in the form of a salt when used for treatment. In some embodiments, the salt is a pharmaceutically acceptable salt.

### VI. Experimental Procedures

TLR-7/8-NF-κB reporter assay. Human embryonic kidney (HEK) cells stably transfected with human TLR-7 or TLR-8 and an NF-κB responsive secreted embryonic alkaline phosphatase (SEAP) gene (Human TLR-7 or 8/NF-κB/SEAP reporter HEK293 cells) can be purchased from InvivoGen (San Diego, CA). The procedure used to measure TLR-7 or TLR-8 agonist activity can be conducted as follows: HEK-TLR-7/8 cells can be grown to 70% to 80% confluency in Normacin, Blasticidin, and Zeocin supplemented DMEM containing 10% HI-FBS. Cells can be seeded in tissue culture-treated flat-bottom 96-well plates at 4×10⁴ cells/well in 100 μL of the above media per well and preincubated for 4 h at 37° C. and 5% CO₂ to allow the cells to adhere to the plate. Concurrently, all compounds can be prepared as 10 mM DMSO solutions and diluted in cell media to a desired initial concentration followed by a series of two-fold dilutions. 100 μL of the media containing diluted compounds can be added to the cell culture in the plates. Alternatively, for competition assays, a known agonist 5 can also be diluted and added to the cells to give final agonist concentrations in 0.1, 0.3, 1, and 3 μM for HEK-TLR-7 cells and 0.3, 1, 3, and 10 for HEK-TLR-8 cells. Following 24 h incubation, 20 μL of the supernatant from each well can be added to 180 μL Quantiblue substrate solution (InvivoGen) and incubated at 37° C. for 15 min. The absorbance at 650 nm was read using a Synergy plate reader (Biotek, Winooski, VT). Data analysis was performed using Prism 6.0 (GraphPad Software, La Jolla California USA). Cell toxicity was not noted for the compounds tested.

Preparation and stimulation of PBMC. Human PBMC can be isolated from heparinized blood by standard density-gradient centrifugation over Ficoll-Paque Plus (Pharmacia, Uppsala, Sweden). Briefly, peripheral blood can be drawn by venipuncture into green top heparin tubes. The blood can be diluted 1:1 with PBS, and then layered upon Ficoll-Paque Plus in 50 ml centrifuge tubes. The tubes can be spun at 2000 rpm for 20 min, after which the buffy coats can be collected and washed twice in PBS. The isolated PBMC can be counted and added to the wells of a 24-well plate (5×10⁵ cells/1 ml/well) in RPMI 1640 supplemented with 10% FBS, penicillin, streptomycin, sodium pyruvate, nonessential amino acids, and HEPES. TLR agonist 5 and TLR antagonist 3 can be prepared in DMSO and added to the wells (in triplicate) to reach the desired final concentrations. Some wells may only contain DMSO. After 24 h, culture supernatants can be collected, aliquoted, and frozen at −80° C. until analysis.

Quantitation of IL-1p, IFNγ, and TNFα. The amount of cytokines present in the supernatants after stimulation can be determined via Enzyme-Linked Immunosorbent Assay (ELISA) using a BioLegend ELISA MAX Deluxe Set for each cytokine tested. The assays can be conducted following the protocols provided by the assay set. Cell culture supernatants from hPBMCs can be used directly without dilution for the determination of IFNγ and TNFα but can be diluted accordingly for the IL-1β so the observed cytokine level could fall into the linear range of the assay.

Provided herein are methods of using one or more compounds of Formula I, conjugate(s) thereof, and/or pharmaceutical compositions comprising such compounds, for the treatment of a disease or condition in a subject in need thereof. In some embodiments, such methods can comprise administering to a subject in need thereof a compound of Formula I, conjugate, or pharmaceutical composition of this disclosure in a therapeutically effective amount to thereby treat such disease or condition in the subject. In some cases, the subject is a mammal such as a human or a rodent. Diseases and/or conditions that can be treated using the compounds of the present disclosure include but are not limited to cancer, a viral or a bacterial infection.

### EXAMPLES

The following examples are given for the purpose of illustrating various embodiments of the invention and are not meant to limit the present disclosure in any fashion. The present examples, along with the methods described herein are presently representative of some embodiments, are exemplary, and are not intended as limitations on the scope of the invention.

Changes therein and other uses which are encompassed within the spirit of the invention as defined by the scope of the claims will occur to those skilled in the art.

### Example 1

### General Synthetic Procedures

This example demonstrates the synthesis of compounds of Formulas I and II.

General Experimental Conditions: Bulk solvents and general chemicals were purchased commercially from Sigma-Aldrich and Fisher Chemical and used without further purification with the exception of THF, which was obtained from a MBraun MB-SPS-800 solvent dispensing system. Moisture- or air-sensitive reactions were conducted under an atmosphere of N₂ or Ar in oven-dried glassware. Reaction progress was monitored by thin layer chromato-graphy (TLC) carried out using Macherey-Nagel Alugram SIL G/UV254 pre-coated sheets. A hand-held UV lamp was used to visualize the plates. A rotary evaporator was used to remove solvents under reduced pressure. Flash column chromatography was performed using a Combi Flash Nextgen 300+ with pre-packed RediSep Rf Gold normal-phase silica gel columns or with SiliaFlash P60 silica gel (40-60 m) purchased from Silicycle. ¹H NMR Spectra were obtained on a Varian 400 MHz or Varian 600 MHz spectrometer in the specified solvent. ¹H NMR peaks are reported as: chemical shift (multiplicity, Jcoupling in Hz, integration). Multiplicity abbreviations used were: s=singlet, d=doublet, t=triplet, q=quartet, quin=quintet, hex=sextet, m=multiplet, br=broad. Mass spectrometry data was obtained on an Agilent liquid chromatography/mass spectrometry (LC-MS) instrument equipped with an ESI interface.

### General Procedure for the Synthesis of Substituted Imidazoles Scaffolds.

Step 1): Aminomalonitrile p-toluenesul-fonate (2.28 g, 9.0 mmol, 1.0 equiv) was suspended in 50 mL of THE at room temperature, followed by the addition of triethylamine (Et₃N) (1.18 g, 11.7 mmol, 1.3 equiv). This mixture was stirred until all the aminomalonitrile p-toluenesulfonate had dissolved to form a homogenous solution. The Orthoester with R¹ (14.4 mmol, 1.6 equiv) was added and the solution was heated at reflux and stirred for 3.5 h. The solution was then cooled to 50° C. and Et₃N (1.18 g, 11.7 mmol, 1.3 equiv) and the primary amine with R² (9.0 mmol, 1.0 equiv) was added. This mixture was stirred at 50° C. for 15 h. Solvent was removed in vacuo and the crude oil was redissolved in 60 mL of dichloromethane (DCM). The solution was washed with saturated aqueous Na₂CO₃ (50 mL) and separated. The aqueous layer was extracted with DCM (3×20 mL). The organic fractions were combined, concentrated in vacuo, and the crude residue was purified by flash chromatography on silica gel.

Step 2): The resulting imidazole (3.0 mmol, 1.0 equiv) was added to a solution of p-TsOH·H₂O (1.71 g, 9.0 mmol, 3.0 equiv) in MeCN (12 mL). This suspension was cooled to 0° C., and to this a solution of NaNO₂ (0.41 g, 6.0 mmol, 2.0 equiv) and KI (1.26 g, 7.5 mmol, 2.5 equiv) in 1.8 mL of water was added dropwise via pipette. This mixture was stirred at 0° C. for 5 min and then allowed to come to room temperature while stirring. This was continued for up to 30 min or until TLC indicated full conversion of the starting material. H₂O (25 mL) was added and sat. aqueous NaHCO₃ was added until the pH reached 9-10 as indicated by pH paper.

Aqueous Na₂S₂O₃ (2 M, 6 mL) was added to remove iodine as NaI, which resulted in a color change of the solution. EtOAc (30 mL) was added and the organic layer was separated from the aqueous layer. The aqueous layer was extracted with three 20 mL portions of EtOAc. The organic fractions were combined, concentrated in vacuo, and the crude residue was purified by flash chromatography on silica gel.

Step 3): The resulting imidazole (3.0 mmol, 1 equiv) was dissolved in CHCl₃ (25 mL). To this mixture was added CH₂I₂(8.84 g, 33.0 mmol, 11 equiv) followed by a solution of isoamyl nitrite (1.93 g, 16.5 mmol, 5.5 equiv) in CHCl₃ (8 ml). This mixture was heated to reflux and stirred for 1 h, turning a darker color over time and evolving a faint brown gas. The mixture was cooled to room temperature, concentrated in vacuo to remove CHCl₃, and the crude residue was purified by flash chromatography on silica gel.

Step 4): Pd₂(dba)3 (66 mg, 0.072 mmol, 0.08 equiv) and SPhos (57 mg, 0.14 mmol, 0.16 equiv) were placed in a flask. Anhydrous THF (3 mL) was added and N₂ was briefly bubbled through the solvent. The mixture was stirred under N₂ at room temperature for 30 min. This catalyst complex mixture was added to a solution of 13 (0.9 mmol, 1.0 equiv), 2-amino-4-methoxycarbonylphenylboronic acid hydrochloride (1.46 g, 6.3 mmol, 1.7 equiv), and Cs₂CO₃ (1.17 g, 3.6 mmol, 4.0 equiv) in THE (20 mL) and H₂O (7 mL), which was flushed with N₂ for 10 min. The reaction mixture was heated to 90° C. and stirred under N₂ for 3 h or until TLC indicated that conversion was complete. The mixture was cooled to room temperature and 20 mL of EtOAc and 15 mL of H₂O were added. The mixture was separated and the aqueous layer was extracted with ethyl acetate (3×15 mL). The organic fractions were combined, concentrated in vacuo and the crude residue was purified by flash chromatography on silica gel.

Step 5): Anhydrous MeOH (3.5 mL) was added to the dried bi-aryl intermediate (0.5 mmol, 1 equiv) in an oven-dried pressure vessel flask and was flushed with argon or N₂. While still under the stream of argon or N₂, 3 pipette drops of conc. sulfuric acid (excess) were added to the reaction mixture. The vessel was capped, and the solution was stirred and heated at 100° C. for at least 5 h. After cooling to room temperature, Na₂CO₃ powder (350 mg) was added to quench the acid. The quenched reaction mixture was dissolved in H₂O (20 mL) and EtOAc (15 mL). The mixture was separated, and the aqueous layer was extracted with 5% MeOH in EtOAc (3×15 mL). The organic fractions were combined and concentrated in vacuo. The crude material was purified by flash chromatography on silica gel. Alternatively, the crude material can be dissolved in a minimal amount of DCM and slowly added hexanes until cloudiness. The solution was then kept at 4° C. overnight to allow crystals of the thiazoquinoline product to form.

### Example 2 Synthesis of methyl 4-amino-1-(2-hydroxypropyl)-2-isobutyl-1H-imidazo[4,5-c]quinoline-7-carboxylate (1)

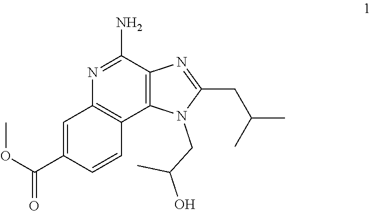 [SMILES: COC(=O)c1ccc2c(c1)nc(N)c1nc(CC(C)C)n(CC(C)O)c12]

The title compound was prepared according to the general procedure of EXAMPLE 1. The product was suspended in 3 mL of DCM, and MeOH was added dropwise until the crude material fully dissolved. To this solution was added hexanes until cloudiness. The solution was cooled to 4° C. overnight and white crystalline solid emerged the next day in 15% yield over two steps. ¹H NMR (400 MHz, DMSO-d₆) δ 8.15 (t, J=1.5 Hz, 1H), 8.08 (d, J=8.6 Hz, 1H), 7.71 (d, J=8.6 Hz, 1H), 6.64 (s, 2H), 5.03 (d, J=5.0 Hz, 1H), 4.59-4.49 (m, 1H), 4.29 (m, 1H), 3.99 (s, 1H), 3.85 (d, J=1.2 Hz, 3H), 2.92-2.73 (m, 2H), 2.26 (m, 1H), 1.22 (d, J=6.1 Hz, 3H), 1.01-0.93 (m, 6H). ¹³C NMR (151 MHz, DMSO-d₆) δ 167.03, 155.02, 152.94, 144.56, 132.41, 128.27, 128.11, 127.27, 121.17, 120.86, 118.74, 65.84, 52.56, 52.50, 36.05, 27.71, 23.01, 22.87, 21.36. [M+H]⁺=299.1872, found 299.1875.

### Example 3 Synthesis of methyl 4-amino-2-(cyclopropylmethyl)-1-(2-hydroxypropyl)-1H-imidazo[4,5-c]quinoline-7-carboxylate (2)

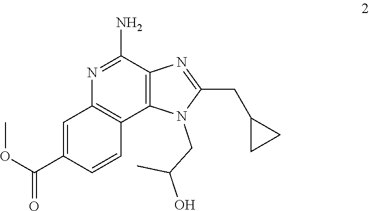 [SMILES: COC(=O)c1ccc2c(c1)nc(N)c1nc(CC3CC3)n(CC(C)O)c12]

The title compound was prepared according to the general procedure of EXAMPLE 1. The product was suspended in 3 mL of DCM and MeOH was added dropwise until the crude material fully dissolved. To this solution was added hexanes until cloudiness. The solution was cooled down to 4° C. overnight and white crystalline solid emerged the next day in 28% yield over two steps. ¹H NMR (400 MHz, DMSO-d₆) δ 8.15 (s, 1H), 8.08 (d, J=8.5 Hz, 1H), 7.71 (d, J=8.7 Hz, 1H), 6.65 (s, 2H), 5.04 (d, J=3.1 Hz, 1H), 4.55 (d, J=15.1 Hz, 1H), 4.30 (dd, J=15.3, 9.0 Hz, 1H), 3.99 (s, 1H), 3.86 (d, J=2.0 Hz, 3H), 3.05-2.78 (m, 2H), 1.28-1.19 (m, 4H), 0.54-0.47 (m, 2H), 0.26 (m, 2H). ¹³C NMR (151 MHz, DMSO-d₆) δ 167.03, 155.33, 152.94, 144.58, 132.53, 128.22, 128.10, 127.34, 121.18, 120.89, 118.70, 65.85, 52.57, 52.56, 31.85, 21.37, 9.56, 5.30, 5.00. [M+H]⁺=355.1770, found 355.1772.

### Example 4 Synthesis of methyl 4-amino-1-(4-(aminomethyl)benzyl)-2-isobutyl-1H-imidazo[4,5-c]quinoline-7-carboxylate (3)

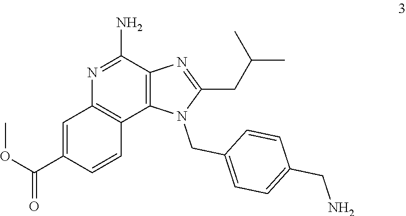 [SMILES: COC(=O)c1ccc2c(c1)nc(N)c1nc(CC(C)C)n(Cc3ccc(CN)cc3)c12]

The title compound was prepared according to the general procedure of EXAMPLE 1. The product was purified by flash column chromatography using a solvent system of 80:14:3:3, EtOAc/MeOH/Et₃N/H₂O, yielding a white solid in 77% yield. Further flash chromatography runs were required to remove minor impurities: R_{f}=0.30 (79:14:4:3, EtOAc/MeOH/Et₃N/H₂O); ¹²H NMR (600 MHz, Methanol-d₄) δ 8.27 (d, J=1.7 Hz, 1H), 7.78 (d, J=8.6 Hz, 1H), 7.58 (dd, J=8.5, 1.8 Hz, 1H), 7.30 (d, J=8.0 Hz, 2H), 7.00 (d, J=8.0 Hz, 2H), 5.85 (s, 2H), 3.90 (s, 3H), 3.73 (s, 2H), 2.85 (d, J=7.4 Hz, 2H), 2.20-2.09 (m, 1H), 0.99 (d, J=6.6 Hz, 6H). MS (ESI+): calcd C₂₄H₂₇N₅O₂ [M+H]⁺418.2238, found 418.2246.

### Example 5 Synthesis of methyl 4-amino-1-(4-(aminomethyl)benzyl)-2-(cyclopropylmethyl)-1H-imidazo[4,5-c]quinoline-7-carboxylate (4)

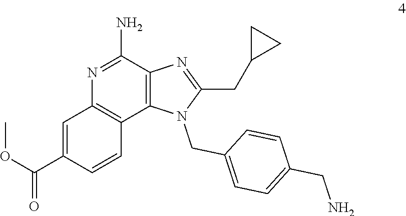 [SMILES: COC(=O)c1ccc2c(c1)nc(N)c1nc(CC3CC3)n(Cc3ccc(CN)cc3)c12]

The title compound was prepared according to the general procedure of EXAMPLE 1. The product was purified by flash column chromatography using a solvent system of 100% EtOAc to 85:9:3:3, EtOAc/MeOH/Et₃N/H₂O, yielding a white solid in 62% yield: R_{f}=0.28 (85:9:3:3, EtOAc/MeOH/Et₃N/H₂O); ¹H NMR (400 MHz, Methanol-d₄) δ 8.30 (d, J=2.3 Hz, 1H), 7.84 (dd, J=8.7, 2.4 Hz, 1H), 7.62 (d, J=8.6 Hz, 1H), 7.36-7.28 (m, 2H), 7.04 (d, J=7.4 Hz, 2H), 5.91 (s, 2H), 3.91 (s, 3H), 3.78 (s, 2H), 2.93 (d, J=6.9 Hz, 2H), 1.22-1.12 (m, 1H), 0.56 (d, J=7.7 Hz, 2H), 0.33-0.25 (m, 2H).

### Example 6 Synthesis of methyl 4-amino-1-(4-(aminomethyl)phenyl)-2-butyl-1H-imidazo[4,5-c]quinoline-7-carboxylate (5)

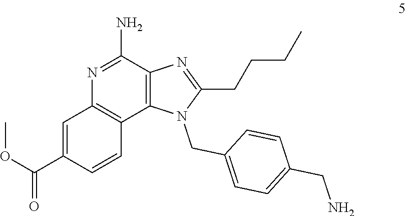 [SMILES: CCCCc1nc2c(N)nc3cc(C(=O)OC)ccc3c2n1Cc1ccc(CN)cc1]

The title compound was prepared according to the general procedure of EXAMPLE 1. The product was purified by flash column chromatography using a solvent system of 100% EtOAc to 94:4:1:1, EtOAc/MeOH/Et₃N/H₂O, yielding a yellow solid in 23.6% yield: R_{f}=0.55 85:9:3:3, 80:10:5:5, EtOAc/MeOH/Et₃N/H₂O); ¹H NMR (CD₃OD, 400 MHz) δ 0.93 (t, J=7.4 Hz, 3H), 1.44 (hex, J=7.4 Hz, 2H), 1.79 (quin, J=7.4 Hz, 211), 2.98 (t, J=7.4 Hz, 2H), 3.79 (s, 2H), 3,90 (s, 3H), 5.88 (s, 2H), 7.03 (d, J=7.8 Hz, 2H), 7.32 (d, J 7.8 Hz, 2H), 7.61 (d, J 9.0 Hz, 1H), 7.83 (d, J=9.0 Hz, 1H), 8.29 (s, 1H). HRMS (ESI³⁰): calcd C₂₄H₂₈N₅O₂ [M+H]⁺418.2238, found 418.2234 (error 0.9 ppm).

### Example 7 TLR-7 and TLR-8 Agonistic and Antagonistic Compounds

This example describes assessment of the TLR-7 and TLR-8 agonist and antagonist activity of compounds 1-5 of the present disclosure.

Compounds 1-5 were screened in vitro for TLR-7 and TLR-8 agonist and antagonist activity using TLR-7 and TLR-8 HEK-SEAP reporter cells and cytokine induction assays. This assay is widely accepted as the standard for determining agonist activity and measures the NF-κB downstream signal mediated by the activation of myeloid differentiation factor 88 (MyD88) by agonist binding.

TABLE 1 below provides the agonist activities as measured for compounds 1-5:

TABLE 1
Reporter Cell Activities for Compounds 1-5^{a}

| Cmpd | TLR-7 | TLR-8 |
|---|---|---|
| 1 | 29.4 ± 2.2 | N/A |
| 2 | 13.4 ± 1.5 | 18.8 ± 3.4 |
| 3^{b} | 3.91 ± 0.15 | 2.19 ± 0.21 |
| 4^{b} | 4.10 ± 0.2 | 4.42 ± 0.46 |
| 5 | 0.18 ± 0.01 | 5.3 ± 0.8 |

^{a}EC₅₀ (μM).
^{b}IC₅₀ (μM) of antagonists (compounds 3 and 4)

As shown in TABLE 1, the N1-hydroxypropyl analogs (compounds 1 and 2) both showed agonist activity for TLR-7 and TLR-8, albeit reduced compared with compound 5.

Additionally, both N1 benzylamine derivatives 3 and 4 were inactive as TLR-7 and TLR-8 agonists.

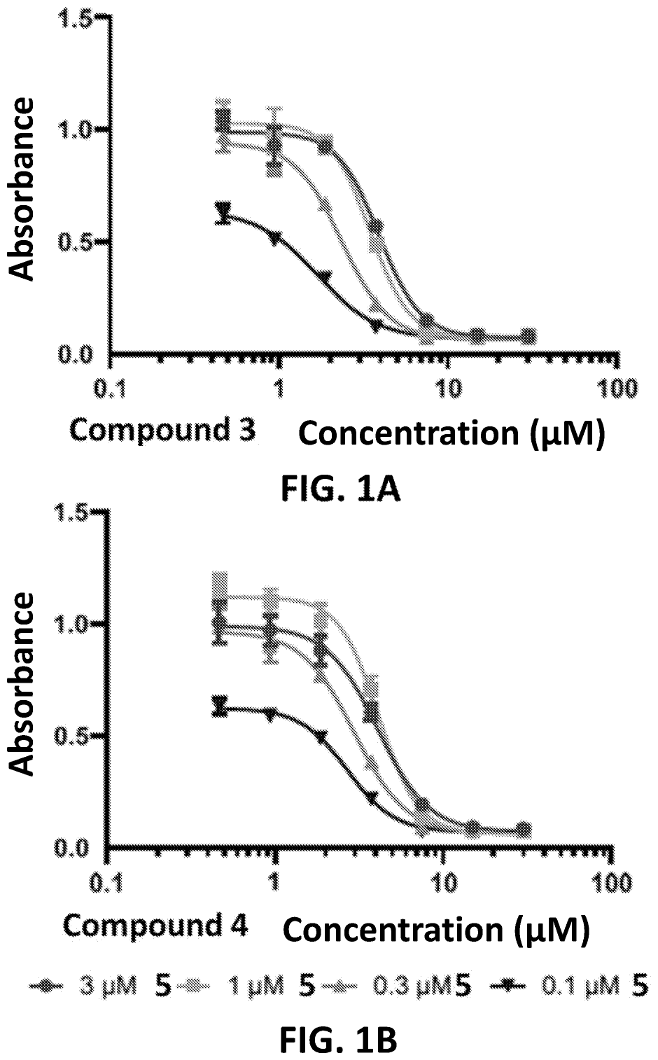

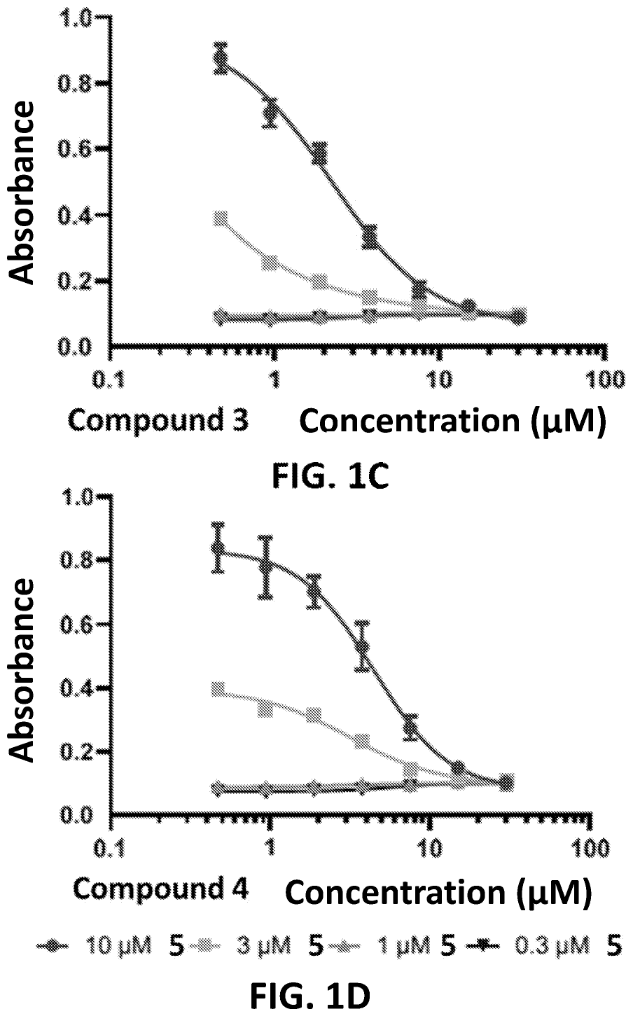

Antagonist screening was performed using the corresponding C2-butyl agonist (compound 5) at three different concentrations. As shown in FIGS. 1A-D, both compounds 3 and 4 produced concentration-dependent shifts to the agonist-induced TLR-7 and TLR-8 dose response curves. The relative IC₅₀ values were also determined at a fixed concentration of compound 5 (3 μM for TLR-7 and 10 μM for TLR-8).

As shown in FIGS. 1A-D, both compounds 3 and 4 are low μM antagonists of compound 5, even at concentrations far above the reported EC₅₀ of the agonist. The N1-benzylamino derivatives (compounds 3 and 4) were found to be highly potent agonists of TLR-7. The finding that compound 3 is capable of inhibiting compound 5 at concentrations more than 10-fold over the EC₅₀ of this agonist was notable and suggested these compounds are near equipotent in binding to the receptor.

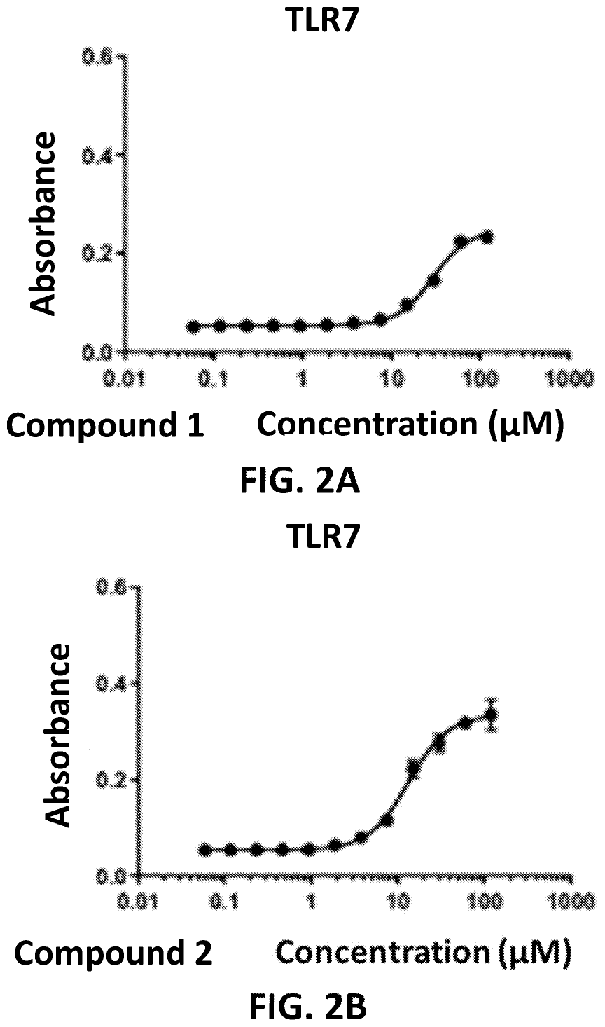

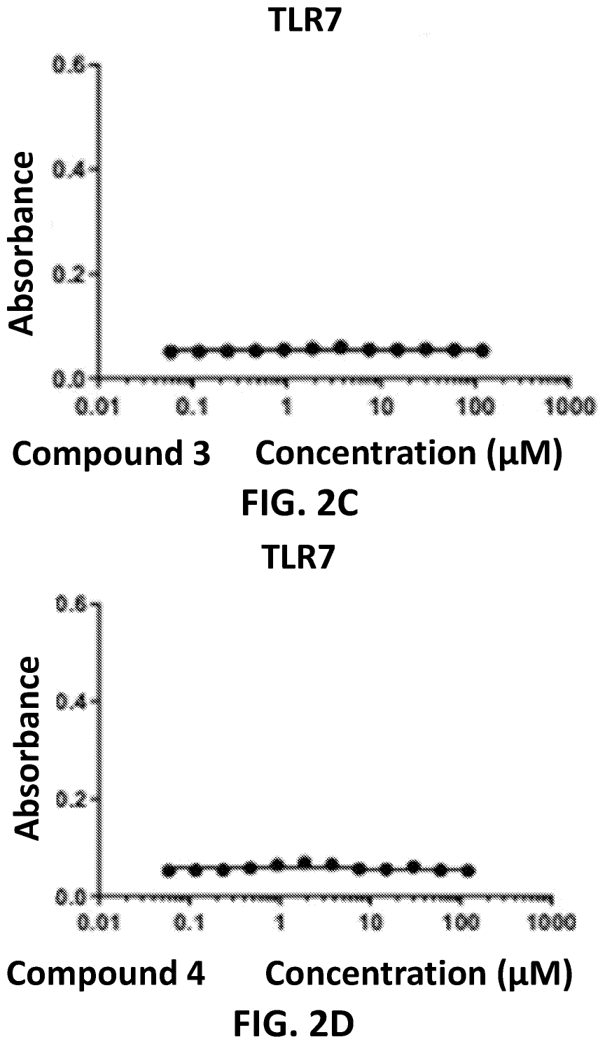

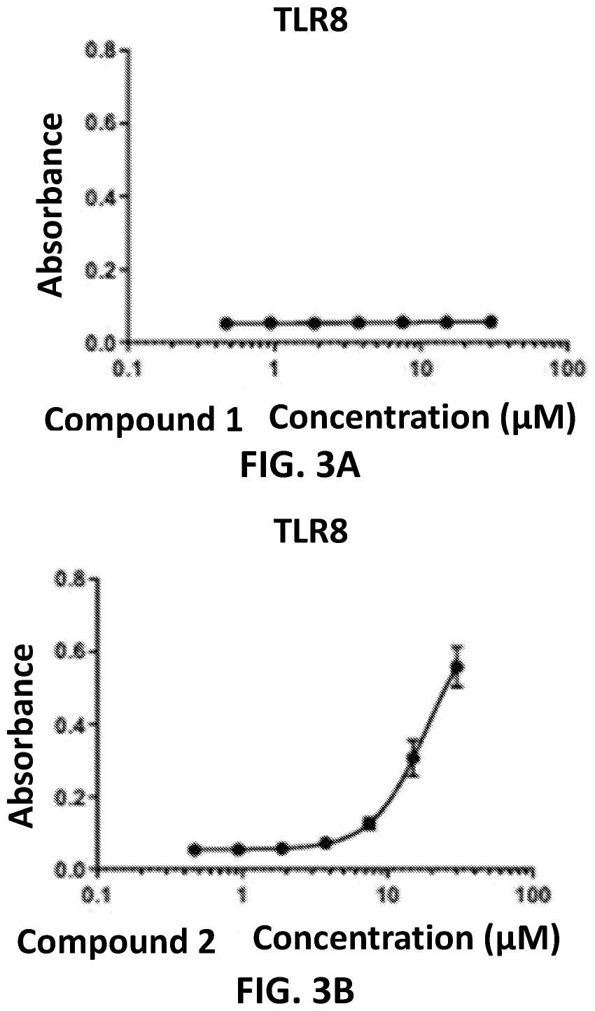

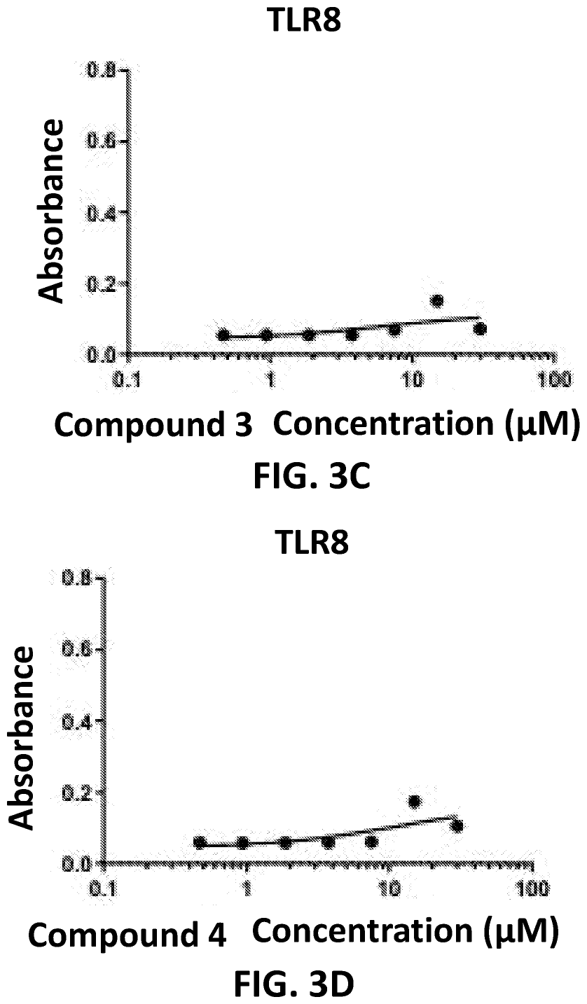

Dose response curves for each of compounds 1-4 at TLR-7 are shown in FIGS. 2A-D. Dose response curves for each of compounds 1-4 at TLR-8 are shown in FIGS. 3A-D. Error bars are included in FIGS. 2A-D and FIGS. 3A-D that represent the standard error of the mean for each point based on the triplicate measurements.

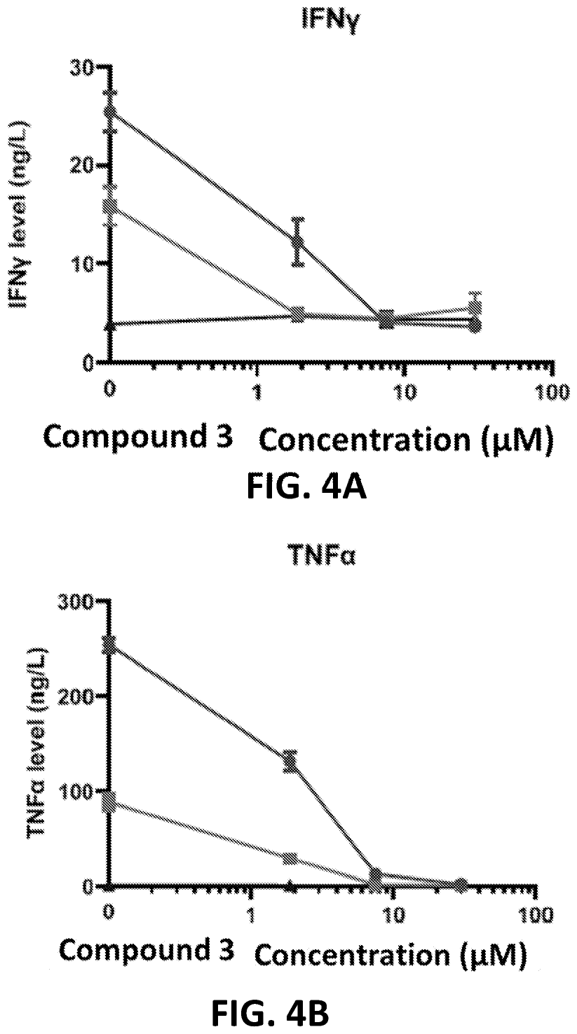

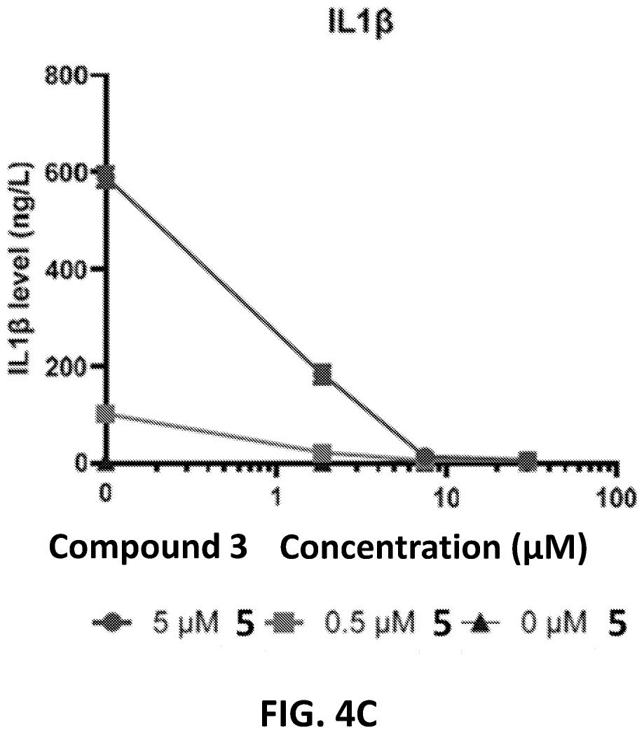

The antagonist activity of compound 4 was further validated using competition assays of agonist activation of cytokine induction in hPBMCs. It was shown that imidazoquinoline (compound 3) is a potent inhibitor of TNFα, IFNγ, and IL-13 production, as shown in FIGS. 4A-C. These cytokines can be of particular interest due to the role they play in the driving Th1 differentiation of immune cells and development of cellular immunity. The results presented in TABLE 1 and FIGS. 1A-D were consistent with the TLR-7 and TLR-8 activation data and showed a concentration-dependent reduction in agonist induction of all three cytokines. Moreover, the data not only confirmed the TLR-7 and TLR-8 HEK-SEAP reporter cell results but also showed that compound 3 seemed to also be devoid of stimulatory activity in hPBMCs.

The results suggest that the C2-position of imidazoquinolines is significant for agonist binding and may also be useful as a chemical switch to flip the conformational equilibrium of the receptor to the antagonist binding state. The C2-substitutent has been shown to project into a hydrophobic pocket at the dimer interface of the ago-nist-bound conformation of the receptor. Hence, it seemed reasonable to conclude the isobutyl and cyclopropylmethyl substitutions disrupt the geometry of the pocket and shift the structural equilibrium to the antagonist conformational state. This finding was surprising and unexpected and suggested that the agonist/antagonist pairs identified in this disclosure can share a close relationship, both in chemical structure and receptor binding and recognition. However, that the potential chemical switch identified here is not universal to imidazoquinoline agonists. The results show the same substitutions merely reduced agonist activity of the N1-hydroxypropyl analog. The effect was more pronounced for the isobutyl derivative (compound 1), which showed a reduction of TLR-8 activity (both as an agonist and antagonist). Without wishing to be bound by any particular theory, it is thought that the inability of compounds 1 and 2 to flip the “switch” suggested the positively charged benzylamino group of 3 and 4 may be significant to both agonist and antagonist binding.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by some embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

What is claimed is:

1. A compound of Formula I: 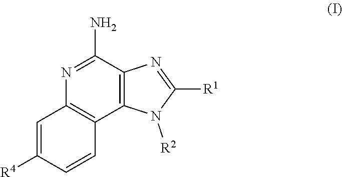
wherein,
R¹, R², and R⁴ are each independently (C₁-C₆)alkyl, (C₂-C₆)alkenyl, (C₂-C₆)alkynyl, (C₁-C₆)alkanoyl, (C₁-C₆)alkoxycarbonyl, (C₁-C₆)alkanoyloxy, (C₃-C₆)-cycloalkyl, aryl, heteroaryl, or heterocycle, which can be optionally substituted with one or more groups independently selected from the group consisting of halo, cyano, hydroxy, oxo, oxiranyl, (C₃-C₅)cycloalkyl, optionally substituted aryl, and optionally substituted heteroaryl,
or a pharmaceutically acceptable salt thereof, and
wherein, when R⁴ is —C(═O)OCH₃ and R² is —CH₂(p-Phe)—CH₂NH₂, R¹ is not —CH₂—CH₂—CH₂—CH₃.

2. The compound of claim 1, wherein R¹ is optionally substituted (C₁-C₆)alkyl.

3. The compound of claim 2, wherein R¹ is —CH₂—CH(CH₃)₂ or

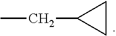

4. The compound of any one of claims 1-3, wherein R² is optionally substituted —CH₂—CH(OH)Me or —CH₂(p-Phe)—CH₂NH₂.

5. The compound of any one of claims 1-4, wherein R⁴ is —C(═O)O—(C₁-C₄)alkyl.

6. The compound of any one of claims 1-5, wherein the compound is selected from the group consisting of:

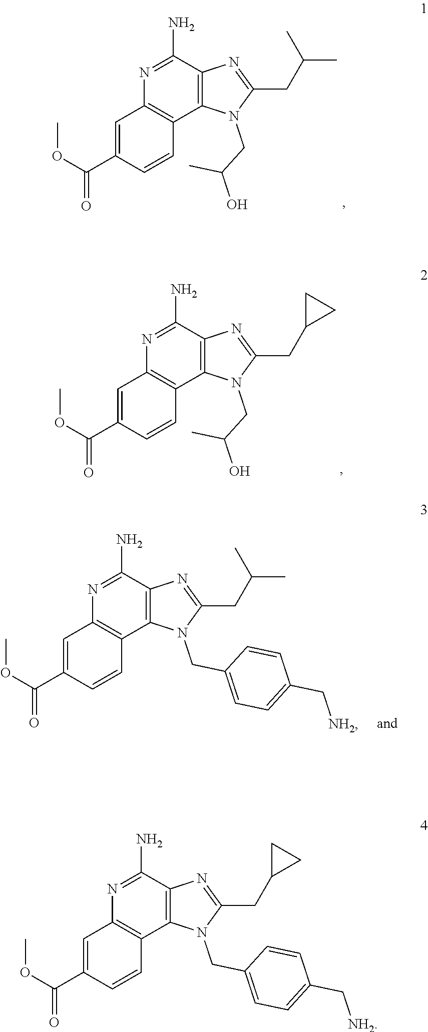 [SMILES: COC(=O)c1ccc2c(c1)nc(N)c1nc(CC(C)C)n(CC(C)O)c12 ; COC(=O)c1ccc2c(c1)nc(N)c1nc(CC3CC3)n(CC(C)O)c12 ; COC(=O)c1ccc2c(c1)nc(N)c1nc(CC(C)C)n(Cc3ccc(CN)cc3)c12 ; COC(=O)c1ccc2c(c1)nc(N)c1nc(CC3CC3)n(Cc3ccc(CN)cc3)c12]

7. A pharmaceutical composition comprising the compound according to any one of claims 1-6 and a pharmaceutically acceptable diluent or carrier.

8. A method of modulating a TLR-7 or TLR-8 receptor, the method comprising contacting the TLR-7 or TLR-8 receptor with the compound of any one of claims 1-6.

9. A method of treating a pathological condition in a subject, the method comprising administering to the subject the compound according to any one of claims 1-6, or a pharmaceutically acceptable salt thereof, or the pharmaceutical composition according to claim 7.

10. A method of stimulating an immune response in a subject, the method comprising administering to the subject the compound according to any one of claims 1-6, or a pharmaceutically acceptable salt thereof, or the pharmaceutical composition according to claim 7.

11. A method of treating a cancer in a subject in need thereof, the method comprising administering to the subject the compound according to any one of claims 1-6, or a pharmaceutically acceptable salt thereof, or the pharmaceutical composition according to claim 7.

12. The method of claim 11, wherein the cancer is multiple myeloma, pancreatic cancer, or lung cancer.

13. A method of treating an autoimmune disease or condition in a subject in need thereof, the method comprising administering to the subject the compound according to any one of claims 1-6, or a pharmaceutically acceptable salt thereof, or the pharmaceutical composition according to claim 7.

14. The method of claim 13, wherein the autoimmune disease is systemic lupus erythematosus (SLE) or rheumatoid arthritis.

15. A method of treating an inflammatory disease or condition in a subject in need thereof, the method comprising administering to the subject the compound according to any one of claims 1-6, or a pharmaceutically acceptable salt thereof, or the pharmaceutical composition according to claim 7.

16. The method of any one of claims 8-15, further comprising administering the subject an immune modulatory therapy.

17. The method of claim 16, wherein the immune modulatory therapy comprises administering a glucocorticoid, a cytostatic agent, an antibody, an immunophilin modulator, a calcineurin modulator, an interferon, an interleukin, a cytokine, or any combination thereof.

18. The method of claim 16 or claim 17, wherein the compound according to any one of claims 1-6, the pharmaceutically acceptable salt thereof, or the pharmaceutical composition according to claim 7 is administered after the immune modulatory therapy.
